(19) United States
(12) Patent Application Publication
(10) Pub. No.: US 2001/0012928 A1
(43) Pub. Date: Aug. 9, 2001

(54) Liquid drug applicator for haired skin

(76) Inventor: Yoko Watabe, Urayasu-shi (JP)

Correspondence Address: Raymond J. Lillie, Esq., Carella, Byrne, Bain, Gilfillan, Cecchi,, Stewart & Olstein, 6 Becker Farm Road, Roseland, NJ, 07068-1739, US

(21) Appl. No.: 09/749,486

(22) Filed: Dec. 26, 2000

(30) Foreign Application Priority Data
Dec. 27, 1999 (JP) 11-377477

(51) Int. Cl. A61M 35/00

(52) U.S. Cl. 604/289

(57) ABSTRACT

The purpose of this invention is to provide an applicator to solve the problems in applying hair tonic or a drug to troubled skin or a hair root on a human scalp or haired skin of pets. Usually, it is difficult to effectively and directly apply a liquid drug to a certain area of troubled skin due to the obstruction by hair on that portion, causing waste of the drug.

Although there have been applicators for coloring bald scalp or scalp with little hair left, an applicator to haired scalp which can push aside the thick hair for direct application to the skin is considered to be the first idea.

For the above-mentioned purpose, on one of the longitudinal sides of this type of a container, a plural number of hollow pipe equipped with the discharge ports respectively are installed in a straight line in the form of comb teeth, and at each discharge port, a portion for contacting the skin is installed, where a groove-like concavity is provided. And the angle of the handle portion extended from the container is formed to have an angle which is convenient for application and enables the whole container to stand up.

### BACKGROUND OF THE INVENTION

1. Field of the Invention

This invention relates to a liquid drug applicator for haired skin, and, particularly to a liquid drug applicator for direct application to the skin.

2. Brief Description of the Prior Art

In case of an irritated haired scalp of a human body, it has been difficult to effectively and directly apply a treatment drug in the form of liquid or cream, due to the obstruction by hair on the skin.

If the troubled portion is small and local, an applicator in the form of a syringe (‘spuit’ in Dutch) can be used. However, such a portion usually covers a wider area, and it is difficult to directly apply a drug to that area without being obstructed by hair. In fact, in many cases, most of the applied drug sticks to hair instead of the troubled skin, which means wasting of a drug and uneconomical. Moreover, it needs the troublesome job of cleaning the hair stained with the drug.

The above-mentioned situation is the same in more daily cases where hair tonic is applied to hair root for preventing baldness. It is neither reasonable nor economical to waste most of the expensive hair tonic by applying it to hair, not to scalp.

The case is quite the same in treatments for troubles of haired skin of cats, dogs and other pets and livestock.

There have been some applicators for scalp for the purpose of coloring the skin to cover the thin hair. But those applicators are for scanty or almost bald scalp.

Accordingly, liquid drug applicators of the present invention for direct application to thick-haired scalp must be the first idea, and it is believed that there has been no prior art to embody such an idea.

As similar applicators for haired skin, there are an aerosol type and a plug type with open/close mechanism, both of which do not work economically and efficiently. For example, in case of the aerosol type, it is difficult to apply liquid directly to scalp unless the hair is pressed down with a hairpin or a hand because the hair is swollen up by its elasticity. In addition, since the discharge port is a little apart from the skin, more liquid is apt to be applied to hair, and only 20-30 percent of the discharged liquid reaches the skin. More than that, the above-mentioned motion must be repeated many times. In case of the plug type with open/close mechanism, the applicator works effectively only for almost bald skin, but not for thick-haired skin. Application with a felt piece is not durable enough in a long-term use.

In the art where a discharge port with a small diameter sealed by the surface tension of the contained liquid is formed and the container itself is pressed to discharge contained liquid, oil, dandruff, and dust are liable to stick to the discharge port and cause the port to be closed, making the liquid discharge difficult. Further, oil, dandruff and dust are liable to enter the container from the discharge port to be mixed with the liquid in it.

In case of an applicator whose tip is the passage of liquid forming a discharge port with a long tubular passage, fresh air cannot easily get into the container from the discharge port, although there is no problem in discharging liquid by pressing the container itself. For example, in case of a common prior art where an applicator body with a line of comb teeth and a handle portion are formed in a straight line, the whole container cannot stand up due to lack of balance, and if the container is left unused for a long time, the surface tension of the contained liquid becomes weaker, causing the liquid to leak out from the discharge port. On the other hand, it is difficult for the contained liquid to flow uniformly to each discharge port at the end of the container from the handle portion.

Thus, in applying a liquid agent to haired skin, a user should do the double jobs in a single motion, that is, pushing aside his thick hair and pressing the container to discharge the liquid, and he should repeat said double jobs many times.

Furthermore, the user should take care so that the liquid, after it is discharged from the port of the container, is applied to his skin evenly, and also he should prevent the liquid from dripping when the discharge port is turned down in use.

### SUMMARY OF THE INVENTION

This invention has been carried out to solve the above-mentioned problems. The purpose of this invention is to provide a means to effectively apply a liquid drug directly to haired skin, without cutting off the hair on the troubled portion. The above-mentioned purpose of this invention is attained by providing a liquid applicator for haired skin, according to either one of the claims 1, 2, 3, 4, 5, 6 or 7.

The following are objectives of the structure of this invention wherein a portion for contacting with the skin, having a similar function to that of the end of the comb tooth, is provided, and a slender, groove-like concavity is installed in the center of the back of the comb tooth:

(1) To push aside the thick hair;

(2) To prevent the discharge port at the end of the applicator from being closed by oil, dandruff and dust;

(3) To prevent oil, dandruff and dust from entering the container from the discharge port to be mixed with the contained liquid;

(4) To make the intake of fresh air from the discharge port easier; and

(5) To realize the uniform application of liquid drug to the skin, by providing a passage for the liquid to be discharged from the discharge port, and even if the discharge port is turned down, the liquid can stay in the container for a little while to prevent dripping, by installing some deep groove-like concavities.

With the five functions mentioned above, said purpose of this invention is to be attained.

As stated in claim 7, the handle portion is extended for a pre-determined length from an end of the container, not in the longitudinally-extended direction from the container, but extended to make an angle which is suitable for liquid drug application to human scalp, and another end of the container is extended to have an expanded bottom surface so that the applicator can stand up. This structure provides the following functions:

(1) To keep the liquid applicator itself standing up with a good balance;

(2) To prevent the leakage of liquid from the discharge port even if the applicator is left unused for a long time;

(3) In use, to enable the contained liquid to flow easily and uniformly to each discharge port at the end of the container from the handle portion, and also to prevent dripping of liquid by reducing the load of air pressure.

(4) To provide the applicator with an angle which makes it convenient for a user to apply liquid to his scalp.

(5) To enable a user to apply liquid in only one motion consisting of two jobs—pushing aside thick hair and pressing the handle portion of an applicator to discharge contained liquid to the port.

These five functions are for attaining the above-mentioned purpose of this invention.

The aforementioned structure of this invention enables a user to apply a required liquid drug quite effectively and directly onto a troubled hair root or scalp in only one motion, without wasting the drug or cutting off any hair in the troubled area.

As mentioned so far, the applicator of this invention is very economical for it realizes an effective and direct application of a required liquid drug to a comparatively wider area on troubled human scalp or an animal's troubled hair root or haired skin. In recent years, common interest in human scalp care is so keen and popular that many TV commercials in that field are often on the air. From such a recent trend, this invention will surely receive strong support by users with its easy and economical operation in use.

### BRIEF DESCRIPTION OF THE DRAWINGS

FIG. 1E is a front view, FIG. 1A is a back view, 1B is a bottom view of the back, 1C is an enlarged cross-sectioned view of the back together with a groove-like concavity in the center of the back of the upper part of the stepwise portion and 1D is an enlarged, cross-sectioned side view of a hollow pipe portion, of an embodiment No. 1.

FIG. 2E is a front view, 2B is a bottom view of the back, and 2D is an enlarged cross section of a side of a hollow pipe portion, of an embodiment No. 2.

FIG. 3E is a front view, FIG. 3A is a back view, FIG. 3B a bottom view of the back and 3D is an enlarged cross section of a side of a hollow pipe portion, of an embodiment No. 3.

FIG. 4E is a front view, 4A is a back view, 4B is a bottom view of the back, and 4D is an enlarged cross section of a side of a hollow pipe portion, of another embodiment No. 4.

FIG. 5A is a cross section of a side, and 5B is a front view, of an embodiment No. 5.

FIG. 5A also shows a cross-sectioned side view and 5B a front view of an embodiment No. 8, wherein a container 1 and a handle portion 1a (extended from a container) are integrated as a whole. And 1b shows a bottom surface of the handle portion (extended from container) of the embodiment No. 8.

Description of Signs

| | |
|---|---|
| 1: | container |
| 1a: | handle portion |
| 1b: | bottom view of handle portion |
| 2: | liquid inlet port |
| 3: | inlet port cover (cap) |
| 4: | hollow pipe |
| 5: | a portion for contacting with skin or scalp |
| 5a: | a liquid discharge port |
| 5b: | a liquid discharge port with a mesh at a part of the lowermost end |
| | of a hollow pipe |
| 5c: | liquid discharge port, equipped with a felt piece F on a part of the |
| | lowermost portion of a hollow pipe |
| 5d: | liquid discharge port utilizing a groove-like concavity |
| 6: | a groove-like concavity |

FIG. 6 shows enlarged figures in moving direction of a pipe end with deep and plural number of groove-like concavity, and an enlarged view of a discharge port.

### DETAILED DESCRIPTION OF THE PREFERRED EMBODIMENTS

Hereunder is the detailed description of the plural number of embodiments according to the Figures attached. [Embodiment No. 1]

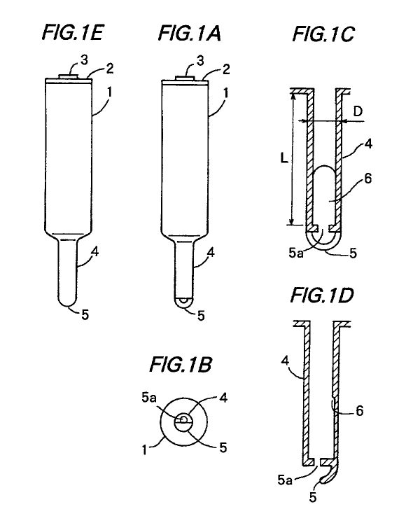

FIG. 1E is a front view, FIG. 1A is a back view, 1B is a bottom view of the back, 1C is an enlarged cross-sectioned view of the back of a hollow pipe portion, with 6 which is a groove in the center of the back of the pipe at an upper portion of the stepwise portion, and 1D is an enlarged cross-sectioned view of a side of the hollow pipe, of an embodiment No. 1 of an liquid drug applicator for direct application to skin according to the present invention. In FIGS. 1E, 1A, 1B, 1C and 1D, 1 is a hollow and cylindrical drug container formed of synthetic resin, for example, equipped with a drug inlet port 2 and a sealing cap 3 at the top. In the lower portion of the container 1, a hollow and comparatively thin pipe 4 having a pre-determined length L with an almost circular cross section is installed to communicate with the container so that the drug in the container 1 can flow into the pipe. The lowermost end of the hollow pipe 4 is formed stepwise with different levels, and a liquid drug discharge port 5a is provided as a passage of liquid at the lower portion of the stepwise part. At an upper portion of the stepwise part, a tip 5 for contacting with skin having a similar function to that of a comb tooth is formed, and in the center of the back of the comb tooth, a slender, groove-like concavity 6 is formed. This embodiment is suitable for portable or disposable uses. Moreover, each constituting member can be selected appropriately. In some cases, the drug discharge port 2 and the cap 3 are not needed; instead, sealing system may be adopted, or a cap may be attached to the hollow pipe 4 for protection.

[Embodiment No. 2]

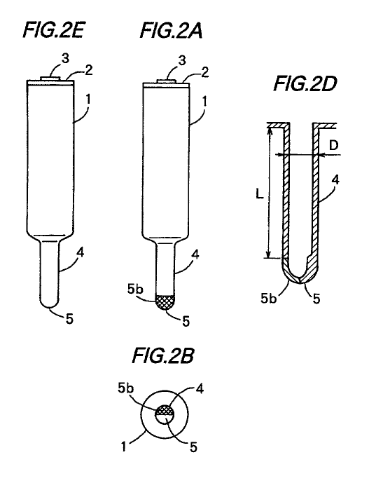

FIGS. 2E, 2A, 2B and 2D show an embodiment No. 2 of this invention, wherein parts corresponding to FIGS. 1A, 1B and 1D of the embodiment No. 1, a front view 2E and a bottom view 2B of the back are shown. At the lowermost end of the hollow pipe 4, a portion 5 for contacting the skin having a similar function to that of a comb tooth tip is installed at a part of the lowermost end of the hollow pipe, and at another part of the lowermost end of the pipe a discharge port 5b equipped with many small holes like meshes is formed. And in the center of the back of the skin-contacting portion a slender, groove-like concavity 6 may be installed.

[Embodiment No. 3]

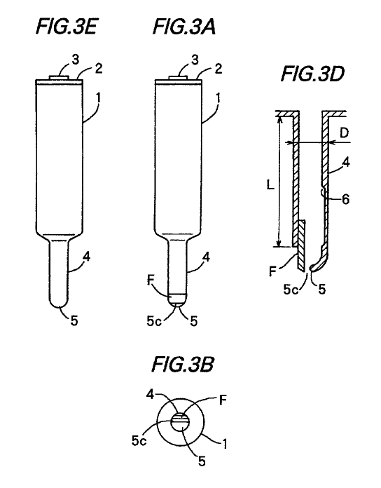

FIGS. 3E, 3A, 3B and 3D show an embodiment No. 3 of this invention, wherein figures corresponding to FIG. 1D of the embodiment No. 1 and FIGS. 2B and 2D of said embodiment No. 2 are shown. At the lowermost end of said hollow pipe 4, a portion 5 for contacting the skin having a similar function to that of a comb tooth tip is installed at a part of the end of the hollow pipe, and in the center of the back of the comb tooth a slender, groove-like concavity 6 is equipped, and at another part of the comb tooth end a discharge port 5c is formed where a permeable, sponge-type felt piece F of a sponge type is installed.

Embodiment No. 4]

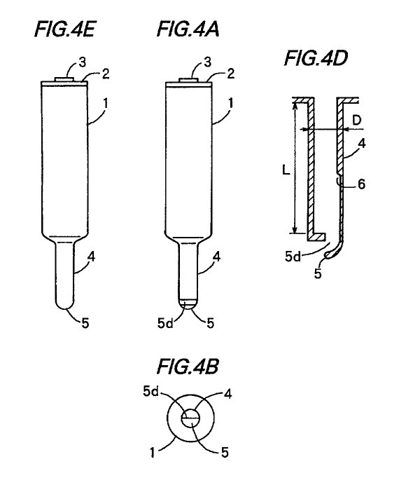

FIGS. 4E, 4A, 4B and 4D show an embodiment No. 4 of this invention, wherein figures corresponding to FIG. 1D of the embodiment No. 1 and FIG. 2E and FIG. 2F of the embodiment No. 2, and an bottom view of an enlarged cross section of the side are shown. The lowermost end of said hollow pipe 4 is formed stepwise with different levels. At the lower portion of the different-leveled end of the pipe, a skin-contacting portion 5 is installed, instead of providing a discharge hole at the lowermost part of the stepwise portion as stated in the embodiment No. 1. In the center of the back of the comb tooth 5, a slender, groove-like concavity 6 is installed. And a space made by the groove-like concavity 6 forms a discharge port 5d.

The difference in the embodiments No. 2, No. 3 and the embodiment No. 4 from the embodiment No. 1 is that 5b of the embodiment No. 2, 5c of No. 3, and 5d of No. 4 are respectively formed as discharge ports, instead of 5a in the embodiment No. 1. Description of other parts is omitted here, as the structures and functions are quite the same as those in the embodiment No. 1.

[Embodiment No. 5]

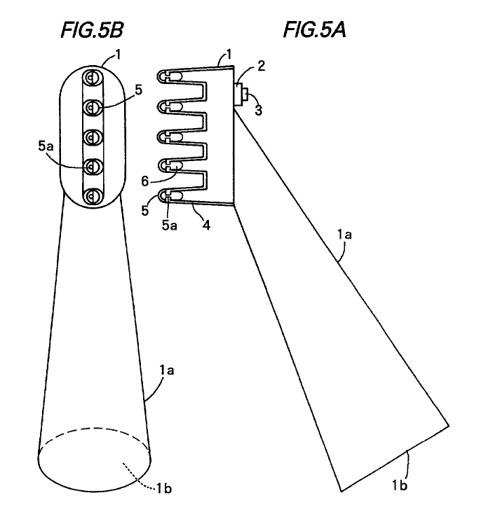

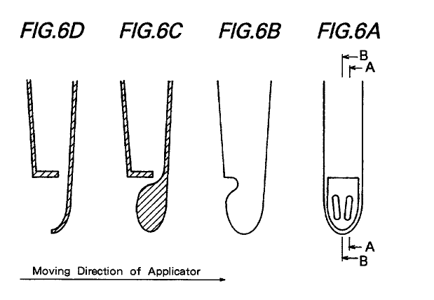

FIGS. 5A and 5B show two views of an embodiment No. 5 of this invention. 5A is a cross-sectioned view of a side, and 5B is a front view with the bottom. Here, description is to be made using the figures of an enlarged cross-sectioned view of a side and a groove-like concavity in the center of the back of the stepwise portion of a hollow pipe and FIG. 1C which were used in the description of the embodiment No. 1. FIG. 5 is also used for the description of the embodiment No. 6 to follow.

In FIGS. 5A and 5B, 1 is a drug container (tank) of an almost rectangular shape, made of a thin rigid resin for example, having a drug-inlet opening (port) 2 on the top, equipped with an appropriate sealing cap 3 of a screwing system or of a push-in system. 4 shows a plural number of synthetic resin hollow pipe for drug application having a pre-determined length and appropriate flexural rigidity, installed with determined spacing in a vertical line in the form of a comb teeth on the front surface of a container. As seen in aforementioned FIG. 1C showing an enlarged cross section of the back of one of the pipes, the hollow pipe is formed to have an almost circular cross section D, a length L, a liquid drug discharge port 5a at the lowermost end of the pipe, a skin-contacting portion 5 and a slender, groove-like concavity 6 are formed. In this embodiment, it is convenient to attach a cap (its figure is omitted here) to cover and protect collectively a plural number of the comb-teeth-shaped hollow pipes 4.

[Embodiment No. 6]

An embodiment No. 6 is to be explained with the two FIGS. 5A and 5B used in describing the embodiment No. 5, and the enlarged cross-sectioned view of a side of a hollow pipe used in the embodiments No. 2, No. 3 and No. 4, as well as FIG. 2D, FIG. 3D and FIG. 4D. The difference of this embodiment from the embodiment No. 5 is that, in FIG. 5A, FIG. 5B and FIG. 1D, the discharge port 5a of the embodiment No. 1 is replaced with the discharge port 5b of the embodiment No. 2, 5c of the embodiment No. 3 and 5d of the embodiment No. 4. Other parts have quite the same structures and functions as those of the embodiment No. 5. Therefore, repeated description is omitted here.

[Embodiment No. 7]

In an embodiment No. 7, the pipes 4 may be installed in the form of ‘a brush’ by repeating the line of the arranged comb teeth, the figure of which is omitted here, although in aforesaid embodiments No. 5 and No. 6 the drug-applying pipes 4 are arranged in a single line like comb teeth.

[Embodiment No. 8]

In an embodiment No. 8, description is made using the liquid container 1 of aforesaid embodiments No. 5, No. 6 and No. 7, and the two figures 5A and 5B with a handle portion 1a (a portion extended from the container) and the bottom surface 1b shown in FIG. 5B. Illustrations of the discharge port 5b, 5c and 5d at the lowermost end of the hollow pipe 4 are omitted. The structures and functions of other portions in the embodiment No. 8 are quite the same as those of other embodiments.

In FIGS. 5A and 5B, the length of the handle portion 1a is determined to suit a user's operation, and the angle of the portion extended for an appropriate length from an end of the container 1 (but not in the longitudinal direction of the container) is determined to be suitable for liquid drug application to human scalp, and another end of the handle portion 1a is extended to expand downward to have the bottom surface 1b. And the container 1 and the handle portion 1a are monolithically formed as a whole with a good balance so that the whole applicator can stand up. Inside the container of this embodiment formed by incorporating the container 1 and the handle portion (extended portion from the container) 1a as a whole, a required liquid drug is contained, and the applicator is structured with the form which enables the whole applicator to stand up without leakage of the contained liquid when it is not used, and, in use the liquid is uniformly discharged from each discharge port 5a, 5b, 5c and 5d by alternatively pressing the opposed side walls of the handle portion 1a. This embodiment is constituted so that the discharge port is not loaded with air pressure. Further, in this embodiment, the applicator is conveniently structured so that the two different jobs—pressing the handle portion 1a and pushing aside the thick hair—are achieved in one motion. In this embodiment, the part of the handle (extended from the container) 1a to be pressed by a user's hand is preferably formed of a synthetic resin having appropriate elasticity.

In above-described embodiments, effective and economical application of a liquid drug directly to troubled skin is realized by determining the size of the container (capacity), and the shapes of the pipe and the discharge port according to required quantity, fluidity and surface tension of liquid drug, hair condition, and/or the width of a troubled portion. More specifically,

(a) The shape of the container is structured so that no air pressure is loaded to a discharge port.

(b) Air pressure at discharge ports is properly distributed according to the number of the comb-teeth (hallow pipes).

(c) A discharge port is formed as a rectangular or an S-shape, instead of a circle with a small diameter.

(d) The slender, groove-like concavity at the end of a comb tooth is made deeper as shown in FIG. 6, and a plural number of groove are installed on it.

(e) The gel-type liquid is more adaptive to various sizes of a discharge port.

The specific examples mentioned above are applicable to a liquid discharge port in each of aforesaid embodiments.

Above-described measures are for cases where liquid is forced to be discharged by pressing the handle portion of a container. However, instead of such types of discharge, an aerosol type, a spray type or a plug type with open/close mechanism may be adopted in production, if it can structurally realize effective application of a liquid drug.

Additionally, containers in above-mentioned embodiments are reusable types in which liquid drug can be refilled. However, in case the production cost for an applicator is low, the container may of course be fabricated as a disposable and/or handy type. If the production cost is high, the number of the comb-tooth (the hollow pipe) may be reduced to only two or three.

What is claimed is:

1. A liquid drug applicator for haired skin, wherein:
a comparatively slender, hollow pipe with a predetermined length is installed on an end of the container so that the liquid can pass through from the container;
the lowermost end of said hollow pipe is installed stepwise with different levels;
at the lowest part of the stepwise portion, a liquid discharge port with a rectangular channel is equipped;
at the apex of the stepwise portion, a part for contacting with skin having a similar function to that of a comb tooth tip is installed; and
in the center of the back of the comb-tooth portion, a single or a plural number of slender, groove-like concavity (or concavities) having a similar function to that of a fountain pen or a pen point, as a discharge port (or discharge ports) of a liquid drug is formed.

2. A liquid drug applicator for haired skin, wherein:
a comparatively slender hollow pipe with a predetermined length is installed on an end of the hollow, cylindrical and sealed container having an almost circular cross section and a pre-determined length, so that the liquid can pass through to the pipe from said container;
a part of the lowermost end of said hollow pipe is formed as a contacting part with skin having a similar function to that of a comb tooth tip; and
another part of the lowermost end of said pipe is formed as a liquid discharge port, where a lot of small holes are installed like meshes.

3. A liquid drug applicator for haired skin, wherein:
a comparatively slender hollow pipe with a pre-determined length is installed on an end of the hollow, cylindrical and sealed container having an almost circular cross section and a pre-determined length, so that the liquid can pass through from said container;
a part of the lowermost end of said hollow pipe is formed as a portion for contacting with skin having a similar function to that of a comb tooth tip;
in the center of the back of the comb-tooth, a single or a plural number of slender, groove-like concavity having a similar function to that of a fountain pen or a pen point is or are installed; and
at another part of the lowermost end of said hollow pipe, said liquid discharge means where a permeable, sponge-like felt piece is installed.

4. A liquid drug applicator for haired skin, wherein:
at the bottom surface of a sealed liquid drug container with a pre-determined length having an almost rectangular or almost circular cross section, a plural number of comparatively slender hollow pipe respectively having pre-determined lengths are installed with pre-determined spacing in a vertical line in the form of comb teeth so that the pipes are communicated with the container and liquid can flow to the pipe from the container;
the lowermost end of each of said hollow pipes is formed stepwise with different levels;
at the lower-leveled portion of each pipe, a liquid discharge port with a rectangular liquid channel is equipped;
at the higher-leveled portion of each pipe, a part for contacting the skin having a similar function as that of a comb tooth tip is installed; and
in the center of the back of the comb-tooth, a single or a plural number of slender, groove-like concavity having a similar function to that of a fountain pen or a pen point is or are installed to work as a liquid discharge port (or as liquid discharge ports).

5. A liquid drug applicator for haired skin, wherein:
at the bottom surface of a sealed liquid drug container with a pre-determined length having an almost rectangular or almost circular cross section, a plural number of comparatively slender hollow pipe respectively having predetermined lengths are installed with predetermined spacing in a vertical line in the form of comb teeth so that the pipes are communicated with the container;
a part of the lowermost end of each hollow pipe mentioned above is formed as a portion for contacting the skin having a similar function to that of a comb tooth; and
another part of the end of the pipe is formed as a liquid discharge port where a number of very small holes are installed in the form of a mesh.

6. A liquid drug applicator for haired skin, wherein:
at the bottom surface of a sealed liquid drug container with a pre-determined length having an almost rectangular or almost circular cross section, a plural number of comparatively slender hollow pipe respectively having pre-determined lengths are installed with pre-determined spacing in a vertical line in the form of comb teeth so that the pipes are communicated with the container and the liquid can pass through to the pipe from the container;
a part of the lowermost end of each hollow pipe mentioned above is formed as a portion for contacting the skin having a similar function to that of a comb tooth tip;
in the center of the back of the comb-tooth tip, a single or a plural number of a slender, groove-like concavity having a similar function to that of a fountain pen or a pen point is or are installed; and
at another part of the end of each pipe, a sponge-like felt piece having permeability (or fluidity) is installed as a liquid discharge means mentioned above.

7. A liquid drug applicator for haired skin according to either one of the claims 4, 5, or 6, wherein:
a length of the container is extended from an end of the container, and the shape of the extended portion is formed to fit a user's hand;
the portion extended from said end of the container for a pre-determined length, but not in the lengthwise direction from the container, is formed to have an angle which is suitable for liquid drug application to human scalp; and
another end of the container is extended to form an expanded bottom surface, and the container and the handle portion (a portion extended from the container) are monolithically formed with a good balance so that the whole container (including the handle portion) can stand up.
